(19) United States
(12) Patent Application Publication
(10) Pub. No.: US 2024/0165264 A1
(43) Pub. Date: May 23, 2024

(54) T CELL ACTIVATION

(71) Applicant: The University of Birmingham, Birmingham (GB)

(72) Inventors: Joseph CLARKE, Birmingham (GB); Alicia EL HAJ, Birmingham (GB); David WRAITH, Birmingham (GB)

(21) Appl. No.: 18/517,633

(22) Filed: Nov. 22, 2023

Related U.S. Application Data
(60) Provisional application No. 63/427,565, filed on Nov. 23, 2022.

(51) Int. Cl. A61K 47/69; A61K 41/00; A61P 37/04; C07K 16/28

(52) U.S. Cl. CPC A61K 47/6929; A61K 41/0052; A61P 37/04; C07K 16/2809; C07K 16/2818

(57) ABSTRACT

Provided is a method of activating T cells, the method including contacting the T cells with a plurality of magnetic nanoparticles (MNPs), wherein each MNP is functionalized with a T cell receptor (TCR) binding moiety, and exposing the T cells to a magnetic field. The method finds utility in the treatment of cancer and autoimmune disease.

### CROSS-REFERENCE TO RELATED APPLICATIONS

This application claims the benefit under 35 U.S.C. § 119(e) of U.S. Provisional Patent App. No. 63/427,565, filed Nov. 23, 2022, the contents of which application are incorporated herein by reference in their entireties for all purposes.

### FIELD OF THE INVENTION

The present invention relates to a method of activating T cells. More particularly, the invention relates to a method of activating T cells by contacting the T cells with magnetic nanoparticles and exposing the T cells to a magnetic field.

### BACKGROUND OF THE INVENTION

The majority of cancer treatments center around surgery and/or chemotherapy, both of which have associated morbidities for patients. Whilst other treatments and immunotherapies for cancers are emerging and show clinical benefit, such as anti-PD-1 treatments and the use of CAR-T-Cell therapies, there is still scope for more targeted cancer therapies within the clinic. Use of small, directed magnetic nanoparticles (MNPs) as a therapeutic holds much benefit for targeted therapies.

Whilst MNP treatments for cancer do exist, they are mainly focused on inducing local temperature increases (hyperthermia) in order to initiate cell death of tumor tissue. Inducing hyperthermia within the cancerous tissue is a non-targeted approach that will induce non-specific cell death of all cells within the cancerous tissue. This is a problem as within the tumor microenvironment are non-malignant, otherwise healthy immune cells that are dysfunctional due to suppression from within the tumor microenvironment. Reactivation of these tumor residing lymphocytes would be of great clinical benefit as it would re-ignite the immune response within the tumor. This direct targeting of specific T cells within the tumor is an unmet need of current cancer therapies.

Many current treatment for autoimmune diseases are non-specific and broadly acting, resulting in numerous side effects including infection and malignant disease. Effective therapies for autoimmune diseases remains a largely unmet clinical need.

The present invention has been devised with these issues in mind.

### BRIEF SUMMARY OF THE INVENTION

In a first aspect of the invention, there is provided a method of activating T cells, the method comprising:
contacting the T cells with a plurality of magnetic nanoparticles (MNPs), wherein each MNP is functionalized with a T cell receptor (TCR) binding moiety; and
exposing the T cells to a magnetic field.

The method may be carried out in vivo, ex vivo or in vitro. In some embodiments, the method is carried out in vivo.

In a second aspect of the invention, there is provided a method of activating T cells in a subject, the method comprising exposing the subject to a magnetic field, wherein the subject has been administered a plurality of magnetic nanoparticles (MNPs), wherein each MNP is functionalized with a T cell receptor (TCR) binding moiety.

In a third aspect of the invention, there is provided a method of activating T cells in a subject, the method comprising:
administering to the subject a plurality of magnetic nanoparticles (MNPs), wherein each MNP is functionalized with a T cell receptor (TCR) binding moiety; and
exposing the subject to a magnetic field.

In a fourth aspect of the invention, there is provided a method of treating cancer or autoimmune disease in a subject, the method comprising exposing the subject to a magnetic field, wherein the subject has been administered a plurality of magnetic nanoparticles (MNPs), wherein each MNP is functionalized with a T cell receptor (TCR) binding moiety.

In a fifth aspect of the invention, there is provided a method of treating cancer or autoimmune disease in a subject, the method comprising:
administering to the subject a plurality of magnetic nanoparticles (MNPs), wherein each MNP is functionalized with a T cell receptor (TCR) binding moiety; and
exposing the subject to a magnetic field.

In a sixth aspect, the invention provides a method of inducing an immune response in a subject, the method comprising exposing the subject to a magnetic field, wherein the subject has been administered a plurality of magnetic nanoparticles (MNPs), wherein each MNP is functionalized with a T cell receptor (TCR) binding moiety.

In a seventh aspect, the invention provides a method of inducing an immune response in a subject, the method comprising:
administering to the subject a plurality of magnetic nanoparticles (MNPs), wherein each MNP is functionalized with a T cell receptor (TCR) binding moiety; and
exposing the subject to a magnetic field.

In any of the methods of the invention, preferably the magnetic field is an oscillating magnetic field. The oscillating magnetic field applies a force to the TCR of a T cell via a bound MNP (which binds to the TCR by virtue of the TCR binding moiety), thereby activating the T cell.

In a further aspect, there is provided a magnetic nanoparticle functionalized with a T cell receptor (TCR) binding moiety.

In another aspect, there is provided a composition comprising a magnetic nanoparticle functionalized with a T cell receptor (TCR) binding moiety, or a plurality of said magnetic nanoparticles.

Also provided is a combination therapy for treating cancer or autoimmune disease, comprising (i) a magnetic nanoparticle (MNP) functionalized with a T cell receptor (TCR) binding moiety and (ii) a further therapeutic agent. The MNP may be one as described herein.

### BRIEF DESCRIPTION OF THE DRAWINGS

Embodiments of the invention will now be described with reference to the accompanying figures in which:

FIG. 1A is a graph showing Nr4a3 expression;

FIG. 1B are graphs showing Nr4a3, CD69 and CD25 expression;

FIG. 2A are graphs showing T cell proliferation, as evidenced by an increase in dilution of CTV proliferation dye as the cell divides, at 3 days post magnetic force treatment;

FIG. 2B are graphs showing the statistical analysis of the CTV traces shown in FIG. 2A;

FIG. 3A shows representative flow cytometry plots showing T cell receptor (TCR) expression for Nr4a3-FT CD4+ T cells;

FIG. 3B is a graph showing the effect of force application on TCR regulation following treatment of T cells with 250 nm anti-CD3 particles or anti-CD3/CD28 (cells that downregulate their TCR become TCRlo);

FIG. 4 shows representative flow cytometry plots showing the effect of Latrunculin A of magnetic force induced TCR downregulation;

FIGS. 5A and 5B are graphs showing Nr4a3 expression within the population of CD4+ cells that are TCRhi and TCRlo as a result of force application;

FIG. 6 are graphs showing surface coating of anti-CD3 antibody on MNPs;

FIGS. 7A to 7C are graphs showing the effect of force application on T cell activation markers Nr4a3, CD69 and CD25 following treatment with MNPs functionalized with different amounts of anti-CD3 antibody;

FIGS. 8A and 8B are graphs showing the effect of MNP surface loading on cell proliferation;

FIGS. 9A and 9B are graphs showing the effect of MNP surface loading on T cell phenotype; and

FIGS. 10A and 10B are graphs showing the effect of force application on Nr4a3 expression over a course of time.

### DETAILED DESCRIPTION OF PREFERRED EMBODIMENTS OF THE INVENTION

The present invention is based on the unexpected finding that T cells can be activated by the application of mechanical force via their T cell receptor (TCR) using magnetic nanoparticles and a remote external magnetic field. This approach holds clinical benefit as it can be used in the clinic in order to drive T cell activation in a targeted manner. For example, in cases where tumor-residing T cells are unable to mount an immune response in the tumor due to the suppressive tumor microenvironment, manipulation of the TCR to drive T cell activation may provide an axis to re-establish anti-tumor immunity and drive tumor clearance. In the context of treating autoimmune disease, functionalization of magnetic nanoparticles with a T cell binding moiety (e.g. major histocompatibility (MHC) proteins loaded with peptides) to target TCRs of specificities known to be involved in autoimmune pathogenesis may provide a means to remotely control T cell phenotype and action.

Provided is a method of activating T cells, the method comprising:
contacting the T cells with a plurality of magnetic nanoparticles (MNPs), wherein each MNP is functionalized with a T cell receptor (TCR) binding moiety; and
exposing the T cells to a magnetic field (e.g. an oscillating magnetic field).

The method may be carried out in vivo, ex vivo or in vitro. For example, in some embodiments the method is an in vivo method for activating T cells in a subject. In such embodiments, the subject is administered, or has previously been administered, a plurality of MNPs, thereby enabling contact between the T cells and the MNPs.

In other embodiments, the method may be carried out ex vivo or in vitro. For example, T cells may be contacted with the MNPs prior to administering the T cells with bound MNPs to a subject. The T cells may be derived from cell culture, from a donor (e.g. a healthy subject) or from the subject to be treated. Thus, in some embodiments the MNPs are bound to T cells prior to administration to the subject.

The subject may be a mammal, such as a human, a primate, a cat, a dog, a horse, a cow, a sheep, a pig, a goat, a rabbit, a mouse or a rat. In some embodiments, the subject is a human.

The invention thus also provides a method of activating T cells in a subject, the method comprising exposing the subject to a magnetic field (e.g. an oscillating magnetic field), wherein the subject has been administered a plurality of magnetic nanoparticles (MNPs) wherein each MNP is functionalized with a T cell receptor (TCR) binding moiety.

Remote activation of the T cells is achieved through the application of an external magnetic field (e.g. an external oscillating or dynamic magnetic field) that induces force application through the TCR. The mechanical movement of the T cell receptor occurs in response to the force of the MNP which moves with the magnetic field gradients and results in mobilization and activation of the TCR. This is evidenced through the downregulation of the TCR, which physiologically occurs at central regions of an immune synapse, termed the central supramolecular activation center (cSMAC). This process requires TCR mobilization. Blockade of TCR downregulation prevents force induced activation marker expression, suggestive of a force induced TCR mobilization mechanism driving T cell activation.

The use of a remote magnetic field for in vivo activation of T cells conveniently avoids the need for in vitro activation and expansion of T cells before administering these to the subject, and instead enables the remote activation of T cells in vivo after administration of MNPs alone.

Thus, in some embodiments the MNPs are not bound to T cells prior to administration to the subject.

In some embodiments, the T cells, or the subject to whom the MNPs have been administered, are exposed to the magnetic field to at least 1 hour. The T cells or the subject may be exposed to the magnetic field for a period of time of from 1 hour to 10 hours, from 2 hours to 8 hours or from 3 hours to 6 hours.

In some embodiments, the T cells or the subject are exposed to a magnetic field of from 30 to 500 mT, from 50 to 300 T, from 100 to 250 mT or from 150 to 200 mT.

The magnitude of the force transferred to each MNP, and thus in turn to the T cells to which they are bound, may be from 10 pN to 500 pN, from 20 pN to 300 pN, from 50 pN to 200 pN or from 150 to 200 pN.

The force can be calculated using formula I:

$${F}_{m a g} = ( {χ}_{2} - {χ}_{1} ) V \frac{1}{{μ}_{0}} B ( ∇ B ) \qquad ( I )$$

wherein χ₂=volume magnetic susceptibility of the magnetic particle attached to the cell,
χ₁=volume magnetic susceptibility of the surrounding medium (i.e. tissue/bone),
μₒ=magnetic permeability of free space, and
B=magnetic flux density in Tesla (T)

It will be appreciated that the number and/or type of magnets, and/or their location relative to the subject or T cells to be treated, can be selected by the skilled person as appropriate in order to achieve the required effect (e.g. the desired therapeutic effect). In some embodiments, the T cells or the subject are exposed to the magnetic field by positioning one or more magnets relative to the T cells or subject such that the T cells are exposed to a desired magnetic field. In some embodiments, the T cells or the subject are exposed to the magnetic field by positioning one or more magnets relative to the T cells or subject such that a desired magnitude of force is transferred to the T cells via the MNPs.

In some embodiments, the subject is exposed to the magnetic field by positioning one or more magnets proximal to, or in contact with, the subject. For example, a magnetic article (e.g. a magnetic bandage or patch, a magnetic strap or sleeve, a magnetic item of clothing or a magnetic blanket) may be applied to the subject, e.g. on or adjacent to the region intended for treatment, such as a tumor site.

In some embodiments the magnetic field is applied using one or more permanent magnets, such as a permanent magnet array. The permanent magnet may be a rare earth magnet. In some embodiments the magnetic field is applied using one or more electromagnets. In some embodiments, the magnetic field is applied using a single or multiple electromagnet arrays. The electromagnetic array(s) may be configured to apply a magnetic field with a magnetic gradient.

Preferably the magnetic field is an oscillating magnetic field. By “oscillating” it will be understood that the magnetic field is not static, for example the gradient of the magnetic field may change over time and/or the magnetic field may be turned on and off. An oscillating magnetic field may be created by moving magnets or by switching them on or off (e.g. rare earth magnets can be moved to change the field strength, or an electromagnet may be turned on and off by a switch). For example, a magnetic array may cycle back and forth with a desired frequency, thereby providing a changing gradient of magnetic forces.

In some embodiments, the magnetic field has an oscillating frequency of from 0.3 to 10 Hz, from 0.5 to 8 Hz or from 1 to 5 Hz. In some embodiments, the magnetic field has an oscillating frequency of 1 Hz.

In some embodiments, the magnetic field is pulsed. By “pulsed”, it will be understood that the magnetic field (e.g. the oscillating magnetic field) is applied in multiple on-off cycles. The magnetic field may be pulsed such that the total time the subject is exposed to the magnetic field for (i.e. the total “on” time) is at least 1 hour. For example, the magnetic field may be applied for at least 4 cycles of 15 minutes on followed by 15 minutes off (i.e. a cycle of on for 15 minutes then off for 15 minutes, repeated 4 times), or at least 2 cycles of at least 30 minutes on followed by 30 minutes off (i.e. a cycle of on for 30 minutes then off for 30 minutes, repeated twice). It will be appreciated that within the “on” phase of a cycle the magnetic field may be rapidly oscillating between on and off states.

In some embodiments, the tissue penetration depth is from 0.5 to 15 cm, from 1 to 12 cm, from 3 to 10 cm or from 5 to 8 cm. It will be appreciated that the depth of tissue within the subject to which the magnetic field penetrates will depend on various factors, including the distance between the subject and the magnet(s). Thus, the subject may be positioned relative to the magnet(s) such that a desired tissue penetration depth is achieved.

In some embodiments, the method comprises administering to the subject the plurality of magnetic nanoparticles (MNPs).

Thus, in an aspect a method of activating T cells in a subject comprises:
administering to the subject a plurality of magnetic nanoparticles (MNPs), wherein each MNP is functionalized with a T cell receptor (TCR) binding moiety; and
exposing the subject to a magnetic field (e.g. an oscillating magnetic field).

The MNPs may be administered by injection, surgical implantation, or orally. In some embodiments the MNPs are administered by injection. For example, the MNPs may be injected into or proximal to a tumor.

The subject may be administered a composition comprising the MNPs. The composition may further comprise a pharmaceutically acceptable excipient or carrier. The pharmaceutically acceptable excipient or carrier may comprise one or more of a buffer, stabilizer, a preservative, a diluent, a disintegrant, a suspending agent, a bulking agent, a salt, a pH-adjusting agent, an adjuvant, a glidant, an emulsifying agent, a wetting agent, and/or a lubricant.

The composition comprising the MNPs may be formulated for injection. Injectable suspensions or solutions may be prepared utilizing aqueous carriers along with appropriate additives.

The invention thus further provides an injectable formulation comprising the MNPs and a pharmaceutically acceptable excipient or carrier.

The methods of the invention may find use in immunotherapy, in particular in the treatment of cancer or autoimmune disease.

Thus, also provided is a method of treating cancer or autoimmune disease in a subject, the method comprising exposing the subject to a magnetic field (e.g. an oscillating magnetic field), wherein the subject has been administered a plurality of magnetic nanoparticles (MNPs), wherein each MNP is functionalized with a T cell receptor (TCR) binding moiety.

Further provided is a method of treating cancer or an autoimmune disease in a subject, the method comprising:
administering to the subject a plurality of magnetic nanoparticles (MNPs), wherein each MNP is functionalized with a T cell receptor (TCR) binding moiety; and
exposing the subject to a magnetic field (e.g. an oscillating magnetic field).

The cancer may be a solid tumor. In some embodiments, the cancer is selected from prostate cancer, breast cancer, lung cancer, colorectal cancer, pancreatic cancer, kidney cancer, liver cancer, a brain tumor, bladder cancer, thyroid cancer, adrenocortical carcinoma, lymphoma, uterine cancer, ovarian cancer, and oral cancer.

The autoimmune disease may be diabetes mellitus, rheumatoid arthritis, psoriasis, psoriatic arthritis, multiple sclerosis, systemic lupus erythematosus (SLE), inflammatory bowel disease (e.g. ulcerative colitis or Crohn's disease), Addison's disease, Graves disease, Sjögren's syndrome, Hashimoto's thyroiditis, Myasthenia gravis, autoimmune vasculitis, pernicious anemia, or celiac disease.

The terms “treating” or “treatment” refer to any indicia of success in the treatment or amelioration of a disease, pathology or condition, including any objective or subjective parameter such as abatement; remission; diminishing of symptoms or making the pathology or condition more tolerable to the subject; slowing in the rate of degeneration or decline; making the final point of degeneration less debilitating; improving the physical or mental well-being of the subject. For example, in relation to the treatment of cancer or autoimmune disease, the treatment may include one or more of the following: cure of the cancer; remission; elimination of tumor(s) or a reduction in tumor size; reduction or elimination of pain, discomfort, or nausea; improvement or restoration of function, such as improved respiratory status or improved motor control; increased lifespan; a reduction in inflammation; improved uptake of nutrients and/or an improvement in the stability of blood sugar levels; increased energy levels; or an improvement in overall wellbeing.

Functionalizing MNPs with a TCR binding moiety provides a mechanism for inducing an immune response, e.g. from within a tumor. The targeted MNPs of the invention can be delivered directly to the tumor where the TCR binding moiety on their surface would be recognized by T cells bearing a TCR that recognizes this moiety. However, this T cell would be unlikely to respond due to the suppressive tumor microenvironment. Remote activation may be achieved via application of an external magnetic field which applies a force to the TCR, which can then re-start the anti-tumor response.

The invention thus also provides a method of inducing an immune response in a subject, the method comprising exposing the subject to a magnetic field (e.g. an oscillating magnetic field), wherein the subject has been administered a plurality of magnetic nanoparticles (MNPs), wherein each MNP is functionalized with a T cell receptor (TCR) binding moiety.

A method of inducing an immune response in a subject comprises:
administering to the subject a plurality of magnetic nanoparticles (MNPs), wherein each MNP is functionalized with a T cell receptor (TCR) binding moiety; and
exposing the subject to a magnetic field (e.g. an oscillating magnetic field).

In some embodiments, the methods of the invention comprise providing a further therapy, e.g. a further anti-cancer therapy or a further autoimmune treatment. The further anti-cancer therapy may include administering a chemotherapeutic agent, hormone therapy, immunotherapy, radiation therapy, photodynamic therapy, stem cell transplant or targeted therapy (e.g. antibody therapy).

The present invention provides a combination therapy for treating cancer or autoimmune disease, comprising (i) a magnetic nanoparticle (MNP) functionalized with a T cell receptor (TCR) binding moiety and (ii) a further therapeutic agent. The MNP may be one as described herein.

In some embodiments the combination therapy is for treating cancer. In such embodiments, the further therapeutic agent may be a an anti-cancer agent, such as a chemotherapeutic agent. Chemotherapeutic agents are well known in the art and include alkylating agents, anti-metabolites, anti-microtubule inhibitors, topoisomerase inhibitors, receptor tyrosine kinase inhibitors, PD-1 inhibitors, angiogenesis inhibitors, and platinum compounds (e.g. carboplatin and cisplatin).

In some embodiments the combination therapy is for treating autoimmune disease. In such embodiments, the further therapeutic agent may be an anti-inflammatory agent (e.g. a nonsteroidal anti-inflammatory medication (NSAID)), a corticosteroid, an analgesic, an immunosuppressant, or an anti-rheumatic drug.

The further therapy or further therapeutic agent may be administered prior to, concurrently or subsequent to administration of the MNPs of the invention to the subject and/or exposing the subject to the magnetic field.

In some embodiments the method further comprises preparing the functionalized MNPs. The MNPs may be functionalized with the TCR binding moiety using any suitable method. In some embodiments, the TCR binding moieties may be conjugated to the MNPs using carbodiimide cross-linking, for example using the methods described herein. In other embodiments, TCR binding moieties may be conjugated to the MNPs using click chemistry (e.g. using the methods described by Thorek et al., Molecular Imaging, Vol. 8, No. 4, pp 221-229).

Methods for generating MNPs will be known to those skilled in the art. Suitable MNPs may also be obtained commercially.

The invention provides a magnetic nanoparticle functionalized with a T cell receptor (TCR) binding moiety.

As is known in the art, magnetic nanoparticles (MNPs) are nanoparticles which can be manipulated using magnetic fields.

The magnetic nanoparticle may have a mean particle diameter of from about 50 nm to about 3 μm, from about 75 nm to about 2 μm, from about 100 nm to about 1.75 μm, from about 200 nm to about 1.5 μm, from about 250 nm to about 1 μm, from about 300 nm to about 750 nm or from about 400 nm to about 500 nm. In some embodiments, the magnetic nanoparticle has a mean particle diameter of from about 100 nm to about 3 μm, or from about 250 nm to about 1 μm. In some embodiments, the magnetic nanoparticle has a mean particle diameter of about 250 nm. In some embodiments, the magnetic nanoparticle has a mean particle diameter of about 1 μm. The size of the MNPs may be determined using a Malvern Zetasizer (e.g. a Malvern Zetasizer NanoZS90) or by Transmission Electron Microscopy (TEM). Methods for preparing and characterizing magnetic nanoparticles are described by Unnithan et al., Adv. Funct. Mater. 2022, 2201311.

The magnetic nanoparticle may be formed from a magnetic element such as iron, nickel or cobalt, an oxide thereof, or a combination thereof. For example, the MNP may comprise magnetite and/or maghemite.

In some embodiments the magnetic nanoparticle comprises one or more rare earth elements such as Dysprosium, Neodymium or Praseodymium.

In some embodiments, the MNPs are superparamagnetic nanoparticles.

In some embodiments the magnetic nanoparticle comprises a surface layer or a coating. The coating may comprise dextran, silica, silicone, a polymer, a precious metal (e.g. gold), graphene or graphene oxide. The surface layer may comprise a metal oxide. For example, a cobalt nanoparticle may have a cobalt oxide surface layer.

The magnetic nanoparticle (or a surface layer or coating thereof) may comprise functional groups (e.g. NH₂, COOH groups) which enable functionalization with the TCR binding moiety.

The magnetic nanoparticle may be directly or indirectly bound to the TCR binding moiety. In some embodiments, the magnetic nanoparticle may be bound to the TCR binding moiety via a linker or a further binding moiety. For example, the magnetic nanoparticle may be functionalized with a capture antibody which in turn is bound to a target antibody that serves as the TCR binding moiety.

Each magnetic nanoparticle may have a surface load of the TCR binding moiety of from 0.5 μg to 20 μg, from 1 μg to 15 μg, from 2 μg to 12 μg, or from 5 μg to 10 μg. The surface load may be determined using methods known to those skilled in the art. For example, qualitative determination of surface load may be carried out by staining the functionalized MNPs using an antibody that binds to the TCR binding moiety on the surface of the MNPs.

In some embodiments, the TCR binding moiety is an antibody.

The term “antibody”, as used herein, will be interpreted broadly as including full length antibodies (i.e. comprise of two heavy chains and two light chains), monoclonal antibodies including murine, human, humanized and chimeric monoclonal antibodies, antigen binding fragments (a portion of an immunoglobulin molecule that binds an antigen, e.g. Fab, F(ab′)₂, Fd and Fv fragments, domain antibodies (dAb) consisting of one VH domain or one VL domain, shark variable IgNAR domains, camelized VH domains, and minimal recognition units consisting of the amino acid residues that mimic the CDRs of an antibody), multispecific antibodies (such as bispecific, trispecific, tetraspecific etc.), dimeric, tetrameric or multimeric antibodies, single chain antibodies, and any other modified configuration of the immunoglobulin molecule that comprises an antigen binding site of the required specificity.

In some embodiments the antibody is an anti-CD3 or anti-CD28 antibody.

In some embodiments, the TCR binding moiety comprises a major histocompatibility complex (MHC)-peptide conjugate. The peptide may be an antigen or epitope (e.g. a known TCR epitope). For example, the peptide may be one which is bound by TCRs of specificities known to be involved in auto-immune responses. The MHC may be a class II molecule.

It will be appreciated that any of the aspects or embodiments described herein can be combined with each other unless otherwise stated.

### EXAMPLES

### Materials and Methods

### Magnetic Particle Functionalization

Commercially available superparamagnetic nanoparticles (MNPs) were purchased with diameters of 250 nm (micromod, 09-02-252) or 1 μm (chemicell, sigmag 1402-1). All commercial particles are a core of iron oxide (maghemite) in a dextran shell coated with surface carboxyl (—COOH) groups to allow for functionalization of biological materials such as antibodies. For functionalization, particles were bound with a capture antibody first via cardobiimide chemical crosslinking, before addition of the target antibody to increase efficiency of target antibody binding. Briefly, 1 mg of particles in 100 μL were first activated in 20 μL of EDAC/NHS solution, freshly prepared each time by dissolving 12/24 mg of sterile EDAC/NHS respectively in 2 ml 0.5 M MES buffer (pH 6.3 in PBS). MNPs were activated in EDAC/NHS by rotation at room temperature for 1 hour. MNPs were then washed three times in 0.1M MES buffer (pH 6.3 in PBS) on a permanent magnet, resuspended in 100 μL 0.1 M MES and were treated with 2 μg of capture antibody unless otherwise stated. MNPs were incubated with capture antibody (AffiniPure Goat anti-Armenian Hamster IgG, Jackson Immunoresearch, 127-005-099) overnight at 4° C. under constant rotation, before three washes as previously described. MNPs were then again resuspended in 100 μL 0.1M MES buffer and treated with 1 μg of target antibody (anti-mouse CD3ϵ, Invitrogen, 16-0031-82, anti-mouse CD28, Invitrogen, 16-0281-82) unless otherwise stated. It should be noted that a ratio of 2:1 capture antibody: target antibody was consistent across all functionalizations. MNPs were incubated with target antibody for 3 hours at room temperature under constant rotation before addition of 10 μL 25 mM glycine in PBS to stop the reaction. MNPs were incubated with glycine for 30 minutes at room temperature under constant rotation, before three final washes as described and resuspension in 0.1% BSA in PBS. All functionalized MNPs were stored at 4° C. for a maximum of 1 week before use in experiments.

### Treatment of CD4+ T cells with Functionalized Magnetic Particles

Splenic tissue was sourced from Nr4a3—fluorescent timer (Nr4a3-FT) reporter mice or from Tg4 transgenic mice, kindly provided by the laboratories of Dr David Bending (University of Birmingham) or Professor David Wraith (University of Birmingham) respectively. CD4+ T cells were purified by negative selection as per the manufacturer's instructions (Invitrogen, magnisort mouse CD4+ enrichment kit, 8804-6821-74) and CD4+ T cell purity was ≥95% in all experiments. Isolated CD4+ T cells were treated with functionalized MNPs for 30 minutes on ice, to final concentrations of 5×10⁵ cells and 50 μg/ml MNPs respectively. Cells were then washed three times in PBS by 5 minutes of centrifugation at 300 g, before resuspension at 1×10⁶/ml and use in force application experiments.

### Force Application Experiments for T cell Activation

Cells treated with MNPs were subjected to external magnetic force through use of the Magnetic Ion Channel Activation (MICA) platform for force application as previously described (Henstock et al., 2018; Hughes et al., 2008). Briefly, the MICA platform permits force application of estimated magnitudes as shown in Table 1, by oscillation of magnetic array beneath the tissue culture plate. In all experiments shown, oscillations were set at 1 Hz and the plate was positioned 3 mm away from the particles in the on position. Force was applied for time frames shown, before assessment of biological T cell activity as described herein.

TABLE 1
Force Estimations for MNPs Used

| Particle | Maximum Force (pN) |
|---|---|
| 250 nm | 1.56 |
| 1 um | 99.846 |

Where indicated, cells were treated with Latrunculin A (Tocris, C973) to final concentrations of 0.25 and 1 μM. Where appropriate, DMSO only controls were included to equivalent an equivalent concentration.

### Flow Cytometry Analysis of T Cell Activation

Following force application for time frames indicated, cells were maintained in a 37° C. 5% CO₂ incubator for 4 hours before assessment of T cell activation by flow cytometry. Nr4a3-FT mice were utilized in these experiments to permit analysis of Nr4a3 expression by following expression of the reporter protein. Cells were surface stained in 100 μL containing antibodies at corresponding dilution factors shown in Table 2. All antibodies for staining were diluted in FACS buffer (PBS+2 mM EDTA+2% FCS), and surface staining was performed at 4° C. for 25 minutes in the dark. Following staining, cells were washed three times by centrifugation at 1800 rpm for 5 minutes, prior to resuspension in 200 μL FACS buffer and data acquisition on a LSR-Fortessa-X20 (BD Bioscience). Post-acquisition analysis was performed in FlowJo (Treestar).

TABLE 2
Surface Antibodies for Flow Cytometry - T Cell Activation Assays

| Target | Fluorophore | Clone | Dilution | Supplier |
|---|---|---|---|---|
| TCRβ | FITC | H57-597 | 1:200 | Biolegend |
| CD4 | AF700 | RM4-5 | 1:200 | Biolegend |
| CD69 | PeCy7 | H1.2F3 | 1:400 | Biolegend |
| CD25 | APC | PC61 | 1:200 | Biolegend |
| Fixable | ef780 | N/A | 1:1000 | Thermo |
| viability dye | (APC-Cy7) | | | Fisher |

For assessment of force application on T cell proliferation, cells were first labelled with cell trace violet (CTV) proliferation dye (thermos fisher, C34557) at a final concentration of 5 μM as per manufacturer's instructions. Following labelling, cells were treated with functionalized MNPs and subjected to force as described. Following force application for 1 hour, cells were maintained in the presence of exogenous 20 U/ml IL-2 (R&D Biosystems, 202-IL) for 72 hours, before assessment of cell proliferation by CTV dilution via flow cytometry. Post-acquisition analysis was performed in FlowJo.

### Flow Cytometry Analysis of T Cell Phenotype

Following force application for 1 hour, cells were maintained for 72 hours in the presence of 20 U/mL IL-2 as described, before restimulation of cells with antigen and antigen presenting cell to assess T cell phenotype. For restimulation, CD4+ T cells were co-cultured with the PL8 antigen presenting cells (Anderton et al., 1998) at both at 1×10⁵/well, and the 4K peptide from myelin basic protein at final concentrations of 10, 1, 0.1 and 0 μg/ml (Liu et al., 1995). Assessment of surface co-inhibitory receptor expression was determined 24 hours post restimulation by surface staining as previously described. Antibodies used in these experiments are detailed in Table 3. Following data acquisition by flow cytometry, post-acquisition analysis was again performed in FloJo.

TABLE 3
Surface Antibodies for Flow Cytometry - T Cell Phenotype Assays

| Target | Fluorophore | Clone | Dilution | Supplier |
|---|---|---|---|---|
| TCRβ | FITC | H57-597 | 1:200 | Biolegend |
| CD4 | AF700 | RM4-5 | 1:200 | Biolegend |
| CTLA-4 | PeCy7 | UC10-4B9 | 1:200 | Biolegend |
| Tigit | PE | 1G9 | 1:200 | Biolegend |
| Fixable | ef780 | N/A | 1:1000 | Thermo |
| viability dye | (APC-Cy7) | | | Fisher |

### Pulsatile Force Application Experiments for NFAT Pathway Accumulation

Purified CD4+ T cells from Nr4a3-FT mice were treated with functionalized MNPs as previously described before application of force as per the following methodologies: 15 mins alone (i.e., in one continuous pulse), 60 mins alone, 15 min×4 (4 pulses of 15 mins of force, each separated by a 15 min break of no force application). For CSA-1, CSA-2, CSA-3 and CSA-4, the calcineurin inhibitor Cyclosporin A (CsA) was added after the first, second, third and fourth pulse respectively to a final concentration of 1 μM. In each setting, the 4 rounds of magnetic treatment were continued to be applied post inhibitor addition to properly assess accumulation of signaling events downstream.

Following force application, Nr4a3 expression was measured by flow cytometry as described 4 hours post force application. Here, surface staining was achieved as previously described and Nr4a3 expression was measured by tracking the fluorescent reporter protein.

### REFERENCES

Anderton, S. M., Manickasingham, S. P., Burkhart, C., Luckcuck, T. A., Holland, S. J., Lamont, A. G., & Wraith, D. C. (1998). Fine specificity of the myelin-reactive T cell repertoire: implications for TCR antagonism in autoimmunity. Journal of Immunology (Baltimore, Md.: 1950), 161(7), 3357-3364.
Henstock, J. R., Rotherham, M., & El Haj, A. J. (2018). Magnetic ion channel activation of TREK1 in human mesenchymal stem cells using nanoparticles promotes osteogenesis in surrounding cells. Journal of Tissue Engineering, 9.
Hughes, S., McBain, S., Dobson, J., & El Haj, A. J. (2008). Selective activation of mechanosensitive ion channels using magnetic particles. Journal of the Royal Society Interface, 5(25), 855-863.
Liu, G. Y., Fairchild, P. J., Smith, R. M., Prowle, J. R., Kioussis, D., & Wraith, D. C. (1995). Low avidity recognition of self-antigen by T cells permits escape from central tolerance. Immunity, 3(4), 407-415.

### Results

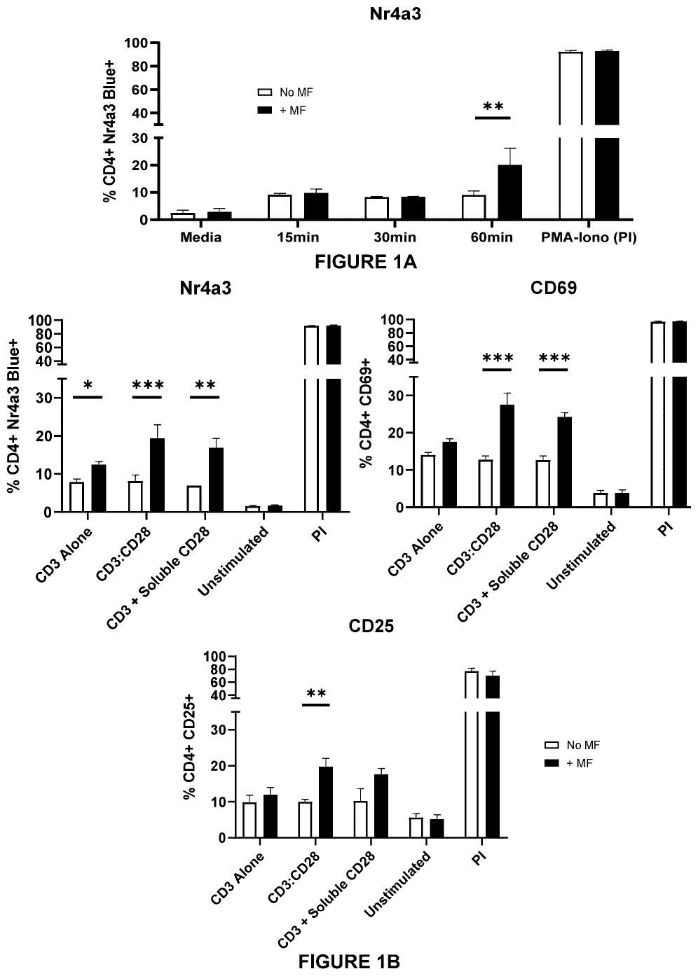

Referring now to FIG. 1A, CD4+ T cells from the Nr4a3-FT mouse were isolated and bound to magnetic nanoparticles (MNPs) functionalized with anti-CD3. External magnetic force was then applied for the times shown before measuring Nr4a3 expression by flow cytometry, 6 hours post force application. Data from 3 independent experiments, statistical significance assessed by 2 way ANOVA with sidak's multiple comparison post tests. **, p=0.0023.

Force application for 1 hour was found to increase the level of naïve CD4+ T cell activation we observed, evidenced by increased Nr4a3 expression, a reporter of TCR signaling in the Nr4a3-FT reporter mouse.

Referring now to FIG. 1B, CD4+ T cells from the Nr4a3-FT mouse were bound to 250 nm particles as described above. Particles were functionalized with anti-CD3, or anti-CD3 and anti-CD28 at a 1:1 ratio, and where indicated anti-CD28 was included soluble in the culture medium at 2 μg/ml. Nr4a3 (FIG. 1B, top left), CD69 (FIG. 1B, top right) and CD25 (FIG. 1B, bottom) expression was assessed 6 hours post force application by flow cytometry. Data from 3 independent experiments, statistical significance assessed by 2 way ANOVA with sidak's multiple comparison post tests. Nr4a3, * p=0.423 *** p<0.0001 ** p=0.0011 (FIG. 1B, top left). CD69, *** p<0.0001, *** p=0.0002 (FIG. 1B, top right). CD25, ** p=0.0041 (FIG. 1B, bottom).

As shown in FIG. 1B, co-stimulation of naïve CD4+ T cells with CD28 stimulation generally increased the level of T cell activation observed (measured by expression of Nr4a3, or activation markers CD69 and CD25). However, importantly effects of magnetic force (black vs white) were driven solely through the TCR-CD3 complex, as the same effect of CD28 stimulation arose when using both bead bound CD28 (CD3:CD28) or soluble CD28 stimulation (CD3+soluble CD28)

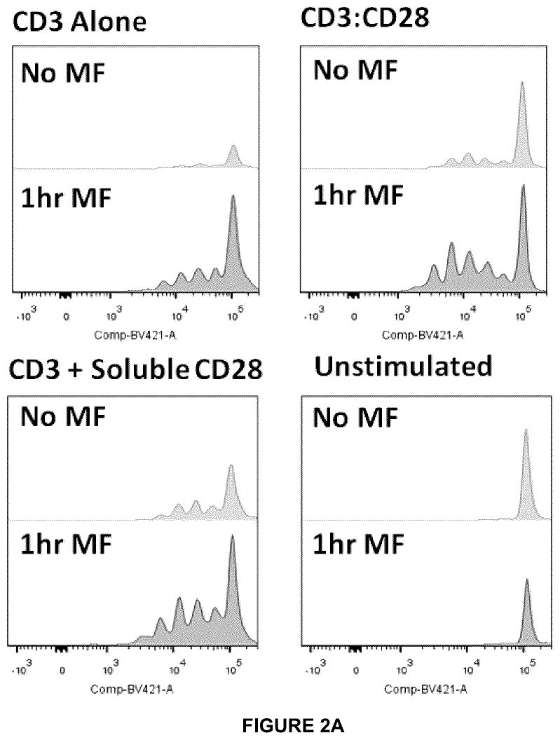

Referring now to FIGS. 2A, CD4+ T cells from the Tg4 transgenic mouse were loaded with 5 μM cell trace violet (CTV) to measure proliferation at 3 days post magnetic force treatment. Force application increases proliferation for all 250 nm particle conditions tested (CD3 alone, top left; CD3:CD28, top right; CD3+ soluble CD28, bottom left; and unstimulated, bottom right).

Referring now to FIG. 2B, there are graphs showing the statistical analysis of CTV traces for CD3 alone (middle), CD3:CD28 (top) and CD3+ soluble CD28 (bottom). For all 3 conditions of 250 nm particle, less cells are found in the undivided peak (division 0) indicating increased cell proliferation. Data from 3 independent experiments, statistical significance assessed by 2 way ANOVA with sidak's multiple comparison post tests. 250 nm CD3 alone (FIG. 2B, middle) division 0 **** p<0.0001. 250 nm CD3:CD28 (FIG. 2B, top) division 0 p<0.0001. 250 nm CD3+Soluble CD28 (FIG. 2B, bottom) ** p=0.0119.

It is shown that force application for 1 hour as per FIG. 1A/B also drives an upregulation in CD4+ T cell proliferation, evidenced by an increase in dilution of the CTV proliferation dye as the cell divides.

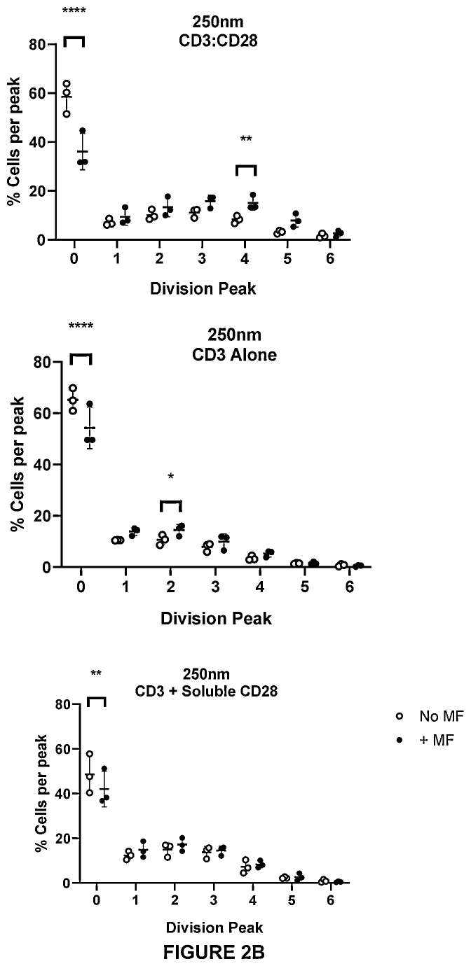

Referring now to FIG. 3A, there is shown representative flow cytometry plots showing T cell receptor (TCR) expression for Nr4a3-FT CD4+ T cells. Cells were stimulated with 250 nm particles as shown, and treated with or without 1 hour of force application. TCR expression was measured by flow cytometry 6 hours post force application.

Referring to FIG. 3B, there is shown a graph displaying force application induced TCR downregulation in response to treatment with 250 nm anti-CD3 particles. Data from 3 independent experiments, statistical significance assessed by 2 way ANOVA with sidak's multiple comparison post tests. Left to right, *** p=0.0003, *** p=0.0009, *** p=0.0002.

FIGS. 3A and 3B show that TCR downregulation occurs under the same kinetics as CD4+ T cell activation, where force application for 1 hour induces robust TCR downregulation (cells that downregulate their TCR become TCRlo).

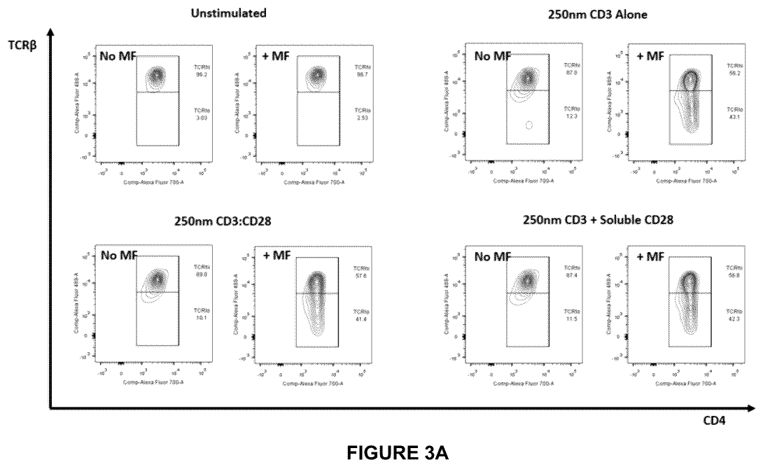

Referring now to FIG. 4, there is shown representative flow cytometry plots showing the effect of Latrunculin A of magnetic force induced TCR downregulation reported in FIG. 3A. Latrunculin A, an inhibitor of actin polymerization, prevents force induced TCR downregulation, showing that the process requires active actin polymerization

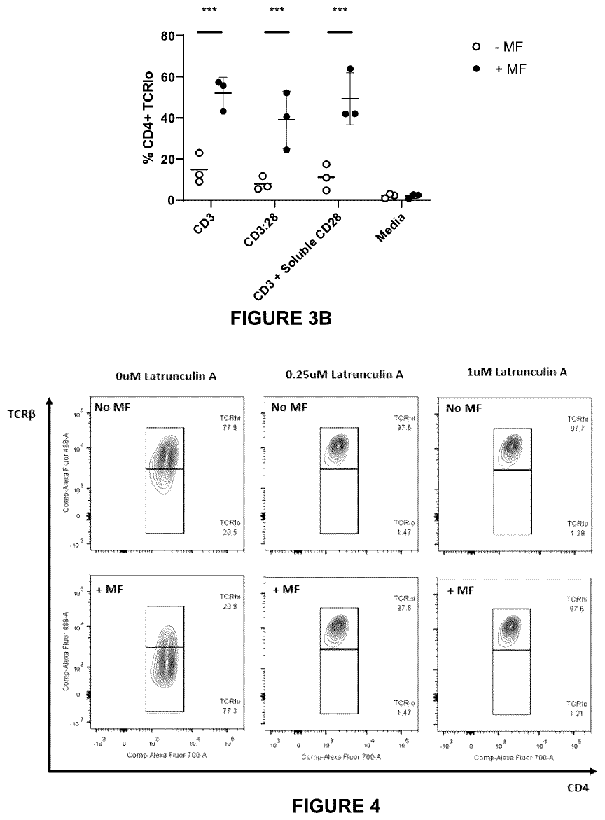

Referring now to FIG. 5A, the graphs show Nr4a3 expression within the population of CD4+ cells that are TCRhi (i.e., do not downregulate their TCR). Within this population, there is no effect of magnetic force on Nr4a3 expression. Data from 3 independent experiments. When looking for effects of force application on Nr4a3 expression in cells that do not downregulate their TCR in response to force, no effect is observed

Referring now to FIG. 5B, the graphs show Nr4a3 expression within the population of CD4+ T cells that are TCRlo as a result of force application (i.e., those that downregulate their TCR in response to force treatment). In the absence of Latrunculin A, force application to both 250 nm CD3 alone particles (left) and 250 nm CD3:CD28 particles (right) increases the number of cells that are TCRlo and expression Nr4a3 (**** p<0.0001, **** p<0.0001). Use of Latrunculin A prevents the establishment of a population of cells that are TCRlo and Nr4a3 positive. Data from 3 independent experiments. Statistical significance assessed by 2 way ANOVA with Sidak's multiple comparison post tests.

Looking in the cells that do downregulate their TCR in response to force application, it is observed that those cells that become TCR in response to force are those that are activating (measured by Nr4ae expression). This process is again blocked by Latrunculin A, which blocks TCR downregulation as per FIG. 4A.

It has thus been shown that force application to the TCR is able to drive T cell activation. Further, force application for 1 hour triggers TCR signaling, driving activation marker expression and ultimately TCR downregulation. Moreover, blockade of actin cytoskeletal dynamics with Latrunculin A prevents TCR downregulation and T cell activation.

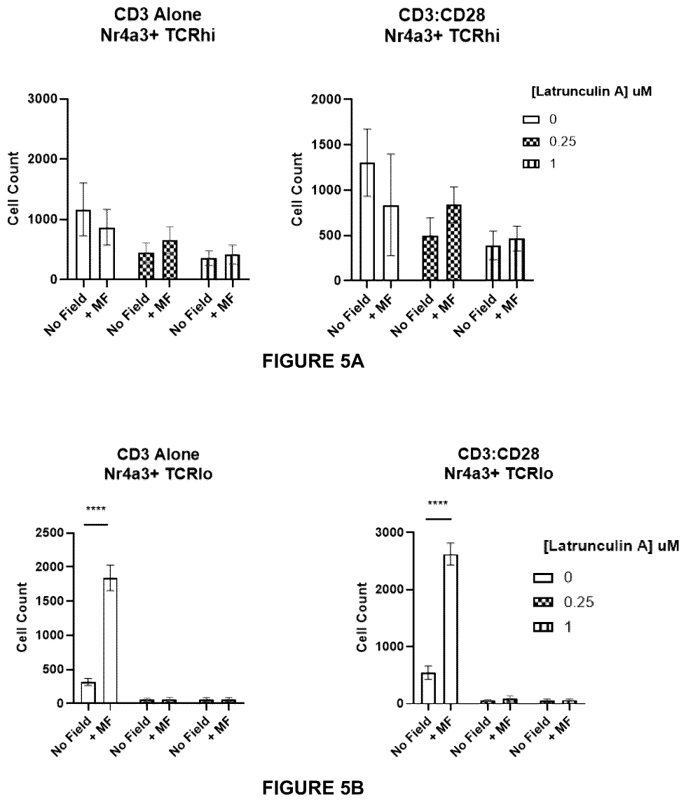

An investigation of surface coating of anti-CD3 on the 1 μm particles is shown in FIG. 6. An anti-Hamster IgG FITC antibody was used to detect the level of CD3 antibody (a Hamster IgG antibody clone) bound to the surface of the particles. FITC fluorescence was detected by flow cytometry. Measurement of the amount of antibody bound to the surface of the 1 μm MNPs shows a clear titration curve.

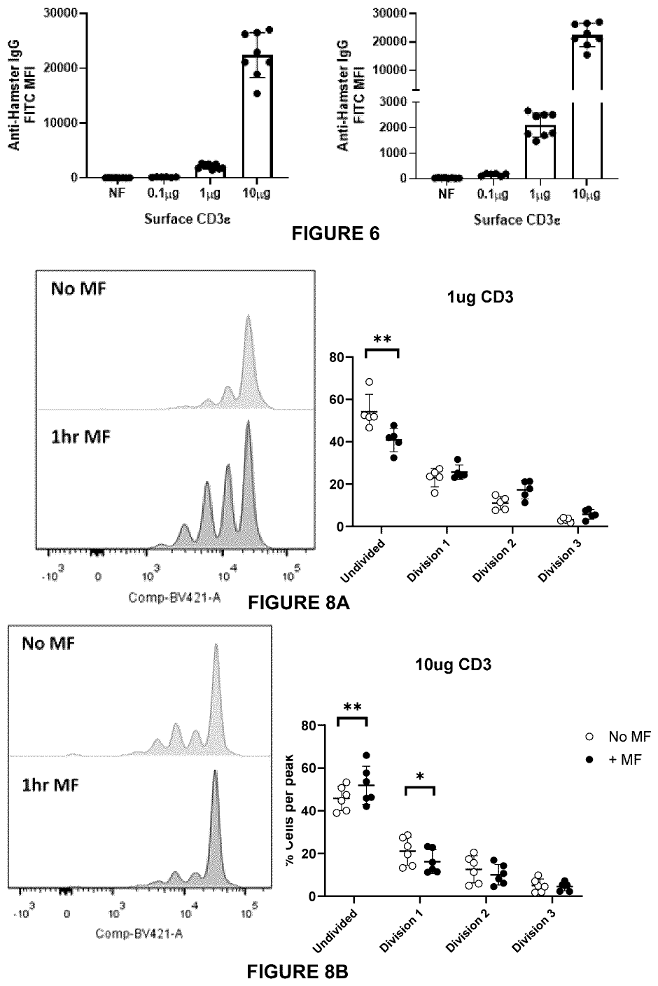

Referring now to FIG. 7A, CD4+ T cells from the Nr4a3-FT mouse were bound to 50 μg/ml 1 μm MNPs, functionalized with the amount of CD3 as shown. Excess particles were removed by PBS washing. External magnetic forces were applied for 1 hour, and Nr4a3 expression was assessed 6 hours post force application. Statistical significance was assessed using 2 way ANOVA with sidak's multiple comparison post tests. *** p<0.0001, ** p=0.0067.

Referring now to FIG. 7B, CD69 expression was measured as per FIG. 7A for Nr4a3. * p=0.0286.

Referring now to FIG. 7C, CD25 expression was measured as per FIGS. 7A and 7B for Nr4a3 and CD69. * p=0.0271, ** p=0.0011.

Force application to 1 μm MNPs functionalized with different amounts of anti-CD3 antibody induces differential effects of TCR activation. For low dose coatings of antibody on the particle surface (1 μg CD3), force application induces upregulation of T cell activation markers Nr4a3, CD69 and CD25. For high dose coatings (10 μg CD3), T cell activation is inhibited as observed through Nr4a3 and CD25 downregulation (FIGS. 7A, 7B, 7C).

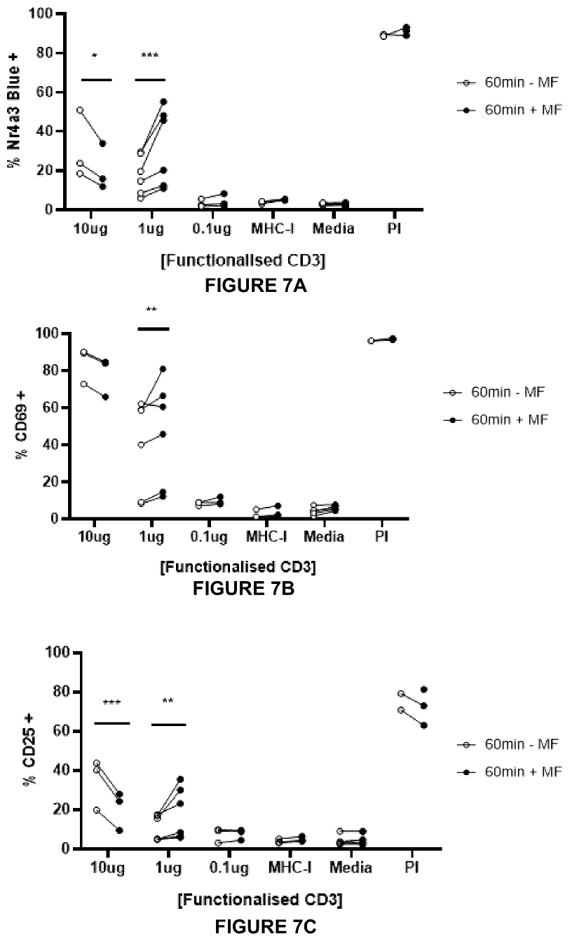

Referring to FIG. 8A, proliferation was measured by CTV dilution to 1 μm particles with 1 μg CD3 as described in FIG. 2A for 250 nm particles. Force application decreases the percentage of cells that remain undivided, indicating that force application to these particles induces cell division. Data from at least 3 independent experiments totaling 5 biological replicates. Statistical analysis measured by 2 way ANOVA with sidak's multiple comparison post tests. ** p=0.0010.

Referring now to FIG. 8B, proliferation was measured by CTV dilution to 1 μm particles with 10 μg CD3 as described in FIG. 2A for 250 nm particles. Force application increases the percentage of cells that remain undivided, indicating that force application to these particles prevents cell division. In line with this, more less cells are then found in division 1. Data from at least 3 independent experiments totaling 6 biological replicates. Statistical analysis measured by 2 way ANOCA with sidak's multiple comparison post tests. ** p=0.0079, * p=0.0347.

It is shown that the differential effects arising from low/high dose coated 1 μm MNPs also translates through to cell proliferation. Low dose coated particles induces an upregulation in T cell proliferation, whilst high dose particles induce a downregulation.

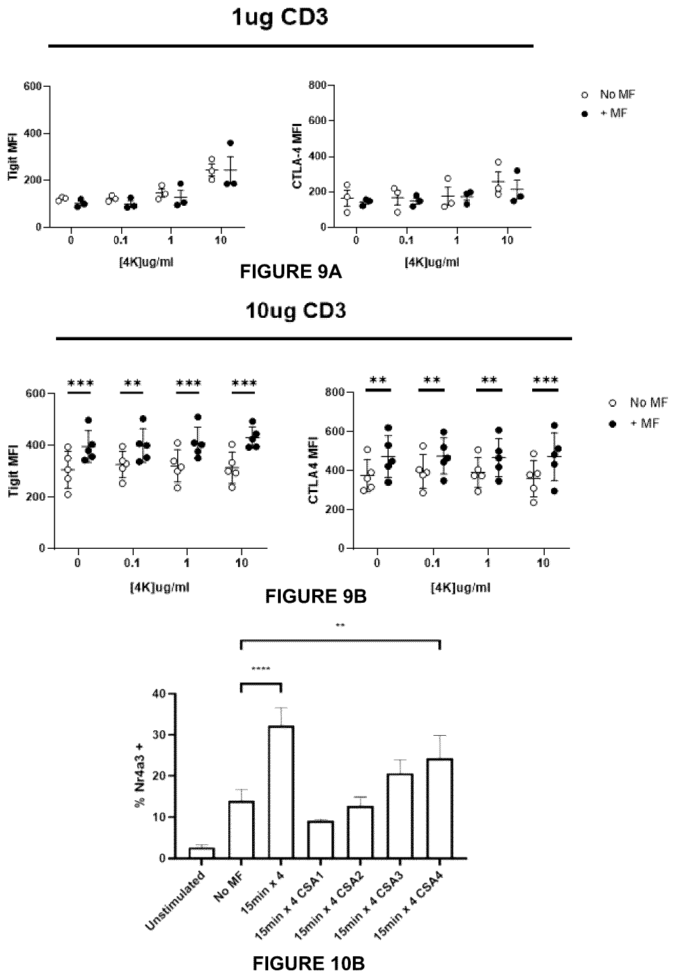

Referring now to FIG. 9A, CD4+ T cells from the Tg4 transgenic mouse were stimulated with 1 μm particles, functionalized with 1 μg as previously described. External force was applied for 1 hour, before cells were restimulated with antigen presenting cells and 4K peptide. 24 hours post restimulation, Tigit and CTLA-4 expression were measured by flow cytometry. External force application has no effect of expression of Tigit or CTLA-4 when cells were stimulated with 1 μm particles functionalized with 1 μg CD3.

Referring now to FIG. 9B, Tigit and CTLA-4 expression as per FIG. 9A, for 1 μm particles functionalized with 10 μg CD3. External force application upregulates expression of both Tigit and CTLA-4 independent of peptide at the point of restimulation, indicating this is driven during the first round of stimulation. Statistical analysis was assessed by 2 way ANOVA with sidak's multiple comparison post tests.

It is shown that differentially coated 1 μm MNPs also induce differential T cell phenotype. Stimulation with low dose coated 1 μm MNPs does not effect expression of co-inhibitory receptors Tigit or CTLA-4, yet use of high dose coated MNPs induces an upregulation in co-inhibitory marker expression when subjected to magnetic force.

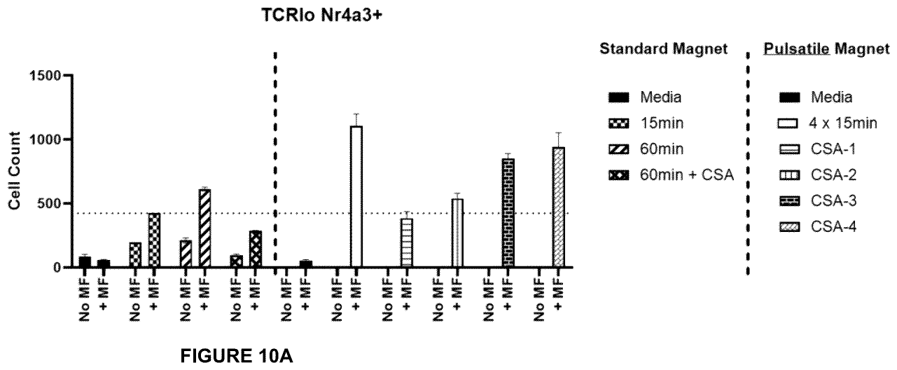

Referring now to FIG. 10A, CD4+ T cells from the Nr4a3-FT mouse were stimulated with 1 μm 1 μg CD3 particles as previously described. External force was applied for the time frames shown. For the standard magnet application, 60 minutes of magnet application is able to drive a population of cells that are TCRlo and expressing Nr4a3.

For the pulsatile magnet pulsing the magnet for 4 rounds of 15 minutes drives a large upregulation of TCRlo NR4a3 expressing cells. For CSA-1-CSA-4, Cyclosporin A (an inhibitor of the calcium-calmodulin-NFAT pathway) was added to the cultures in a sequential manner, to a final concentration of 1 uM. CSA-1 was added after the first 15 min pulse, CSA-2 after the second, CSA-3 after the third and CSA-4 after the fourth. The data indicates that pulsatile magnet application for rounds of 15 minute pulses drives an accumulation of NFAT activity which drives Nr4a3 expression. Data from 3 independent experimental repeats.

Referring now to FIG. 10B, pulsatile magnet stimulation was applied as described for FIG. 10A. Use of the calcineurin inhibitor cyclosporin A (CsA) prevents an accumulation of signaling events across the NFAT pathway over course of 1 hour magnetic field application. Data from 3 independent experiments, statistical analysis performed via one-way ANOVA with Dunnett's multiple comparisons comparing all conditions to no magnetic force (MF) control. **** p<0.0001, **, p-0.0085. Not shown: Unstimulated vs No MF, **, p=0.0038.

It is shown that force application to 1 μm MNPs coated with the low dose of CD3 (1 μg) induces an accumulation of signaling events in the NFAT pathway across the course of 1 hour. Blocking the NFAT pathway with CsA after the 1st, 2nd, 3rd or 4th 15 min pulse shows a clear time response for Nr4a3 expression across this time course.

What is claimed is:

1. A method of activating T cells, the method comprising:
contacting the T cells with a plurality of magnetic nanoparticles (MNPs), wherein each MNP is functionalized with a T cell receptor (TCR) binding moiety; and
exposing the T cells to a magnetic field.

2. The method of claim 1, wherein the magnetic field is an oscillating magnetic field.

3. The method of claim 1, wherein the T cells are exposed to the magnetic field to at least 1 hour.

4. The method of claim 1, wherein the magnetic field is pulsed.

5. The method of claim 1, wherein the TCR binding moiety comprises an antibody or a MHC-peptide complex.

6. The method of claim 1, wherein the MNPs have a mean particle diameter of from 100 nm to 3 μm.

7. The method of claim 1, wherein each MNP has a surface load of the TCR binding moiety of from 0.5 μg to 20 μg.

8. The method of claim 1, wherein the method is carried out in vivo.

9. The method of claim 1, wherein the method is for activating T cells in a subject, the method comprising exposing the subject to the magnetic field, wherein the subject has been administered the plurality of magnetic nanoparticles (MNPs).

10. The method of claim 9, wherein the method is for treating cancer or autoimmune disease.

11. The method of claim 9, wherein the method further comprises administering to the subject the plurality of magnetic nanoparticles (MNPs).

12. The method of claim 11, wherein the MNPs are not bound to T cells prior to administration to the subject.

13. The method of claim 11, wherein the MNPs are administered by injection, optionally wherein the MNPs are injected into or proximal to a tumor.

14. The method of claim 9, wherein the method comprises administering a further therapeutic agent.

15. The method of claim 1, wherein the method further comprises functionalizing MNPs with the TCR binding moiety.

16. A method of treating cancer or autoimmune disease in a subject, the method comprising:
administering to the subject a plurality of magnetic nanoparticles (MNPs), wherein each MNP is functionalized with a T cell receptor (TCR) binding moiety; and
exposing the subject to a magnetic field.

17. The method of claim 16, wherein the magnetic field is an oscillating magnetic field.

18. A magnetic nanoparticle functionalized with a T cell receptor (TCR) binding moiety.

19. The magnetic nanoparticle of claim 18, wherein the nanoparticle has a mean particle diameter of from 100 nm to 3 μm and/or wherein the nanoparticle has a surface load of the TCR binding moiety of from 0.5 μg to 20 μg.

20. The magnetic nanoparticle of claim 18, wherein the TCR binding moiety comprises an antibody or a MHC-peptide conjugate, optionally wherein the antibody is an anti-CD3 or anti-CD28 antibody.
